(19) United States
(12) Patent Application Publication
(10) Pub. No.: US 2021/0251517 A1
(43) Pub. Date: Aug. 19, 2021

(54) UNOBSTRUSIVE ESTIMATION OF CARDIOVASCULAR PARAMETERS WITH LIMB BALLISTOCARDIOGRAPHY

(71) Applicant: University of Maryland, College Park, College Park, MD (US)

(72) Inventors: Jin-Oh Hahn, Potomac, MD (US); Yang Yao, Changtu County (CN); Sungtae Shin, Greenbelt, MD (US); Azin Sadat Mousavi, College Park, MD (US)

(21) Appl. No.: 17/176,166

(22) Filed: Feb. 15, 2021

Related U.S. Application Data
(60) Provisional application No. 62/976,883, filed on Feb. 14, 2020.

(51) Int. Cl. A61B 5/11; A61B 5/024; A61B 5/00; A61B 5/021; A61B 5/02

(52) U.S. Cl. CPC A61B 5/1102; A61B 5/02427; A61B 5/725; A61B 5/7285; A61B 5/021; A61B 5/02028; A61B 5/266; A61B 5/6826; A61B 5/6824; A61B 5/02108; A61B 5/6831; A61B 2562/0219; A61B 2562/0261; A61B 5/02007

(57) ABSTRACT

Aspects of the disclosure relate to estimation of cardiovascular parameters based on a ballistocardiogram signal. In one example, an apparatus for estimating cardiovascular parameters includes a BCG sensor for producing a BCG signal of a user, a processor, a display, and a memory communicatively coupled to the processor. The processor and the memory are configured to transform the BCG signal to a synthetic whole-body BCG signal by integrating the BCG signal in time twice and zero-phase filtering the BCG signal, estimate the cardiovascular parameters based on the synthetic whole-body BCG signal, and display the cardiovascular parameters to the display. Other aspects, embodiments, and features are also claimed and described.

### CROSS-REFERENCE TO RELATED APPLICATIONS

This application claims priority from U.S. Provisional Patent Application No. 62/976,883, filed Feb. 14, 2020, which is incorporated herein by reference in its entirety and for all applicable purposes.

### TECHNICAL FIELD

The technology discussed below relates generally to estimating cardiovascular (CV) parameters using a ballistocardiogram (BCG) signal.

### INTRODUCTION

Cardiovascular disease (CVD) is a leading cause of mortality and morbidity that produces immense health and economic impacts in the United States and globally. The measurement of clinically significant CV parameters often requires inconvenient instruments and even invasive procedures. For example, the gold standard arterial blood pressure (BP) waveform is measured by invasive arterial catheterization. There are non-invasive options such as volume clamping techniques and applanation tonometry, but these techniques require costly equipment and/or trained operators. The gold standard stroke volume (SV), cardiac output (CO), and total peripheral resistance (TPR) likewise require inconvenient and costly procedures such as dye injection, echocardiography, impedance cardiography, and electrical impedance tomography. The CV parameters have also been derived indirectly using the so-called pulse contour methods. These methods have been extensively investigated and demonstrated success. Yet, the techniques still necessitate the invasive or inconvenient measurement of arterial BP waveforms. Considering the prevalence and implications of CVD on the quality of life and healthcare cost, non-invasive and convenient measurement of clinically significant cardiovascular (CV) parameters plays an important role in effective prevention and treatment of CVD.

### BRIEF SUMMARY OF SOME EXAMPLES

The following presents a simplified summary of one or more aspects of the present disclosure, to provide a basic understanding of such aspects. This summary is not an extensive overview of all contemplated features of the disclosure, and is intended neither to identify key or critical elements of all aspects of the disclosure nor to delineate the scope of any or all aspects of the disclosure. Its sole purpose is to present some concepts of one or more aspects of the disclosure in a simplified form as a prelude to the more detailed description that is presented later.

In various aspects, the disclosure generally relates to estimating CV parameters based on a BCG signal. In some scenarios, an apparatus may include a BCG sensor for producing a BCG signal of a user, a processor, a display, and a memory communicatively coupled to the processor. The processor and the memory are configured to: transform the BCG signal to a synthetic whole-body BCG signal by integrating the BCG signal in time twice and zero-phase filtering the BCG signal, estimate the cardiovascular parameters based on the synthetic whole-body BCG signal, and display the cardiovascular parameters to the display.

These and other aspects of the technology discussed herein will become more fully understood upon a review of the detailed description, which follows. Other aspects, features, and embodiments will become apparent to those of ordinary skill in the art, upon reviewing the following description of specific, exemplary embodiments in conjunction with the accompanying figures. While the following description may discuss various advantages and features relative to certain embodiments and figures, all embodiments can include one or more of the advantageous features discussed herein. In other words, while this description may discuss one or more embodiments as having certain advantageous features, one or more of such features may also be used in accordance with the various embodiments discussed herein. In similar fashion, while this description may discuss exemplary embodiments as device, system, or method embodiments it should be understood that such exemplary embodiments can be implemented in various devices, systems, and methods.

### BRIEF DESCRIPTION OF THE DRAWINGS

FIG. 1 is a block diagram conceptually illustrating a BCG analysis procedure according to some embodiments.

FIG. 2 illustrates instrumented physiological signals according to some embodiments.

FIG. 3 is a flow chart illustrating hemodynamic interventions according to some embodiments.

FIG. 4 is a block diagram conceptually illustrating a signal preconditioning procedure according to some embodiments.

FIG. 5 is a block diagram conceptually illustrating a procedure for transforming armband BCG to whole-body BCG according to some embodiments.

FIG. 6 illustrates representative whole-body and armband BCG waveforms according to some embodiments.

FIG. 7 illustrates group-average changes in the CV parameters in response to hemodynamic intervention according to some embodiments.

FIG. 8 illustrates correlation plots between measured versus regressed CV parameters associated with a whole-body BCG according to some embodiments.

FIG. 9 illustrates Bland-Altman plots between measured versus regressed CV parameters associated with a whole-body BCG according to some embodiments.

FIG. 10 illustrates correlation plots between measured versus regressed CV parameters associated with a synthetic whole-body BCG according to some embodiments.

FIG. 11 illustrates Bland-Altman plots between measured versus regressed CV parameters associated with a synthetic whole-body BCG according to some embodiments.

FIG. 12 is a schematic illustration of the relationship between characteristic features in a BCG signal and CV parameters according to some embodiments.

FIG. 13 is a block diagram conceptually illustrating an example of a hardware implementation for a scheduling entity according to some embodiments.

FIG. 14 is a flow chart illustrating an exemplary process for estimating CV parameters based on an armband BCG signal according to some embodiments.

### DETAILED DESCRIPTION

The detailed description set forth below in connection with the appended drawings is intended as a description of various configurations and is not intended to represent the only configurations in which the concepts described herein may be practiced. The detailed description includes specific details for the purpose of providing a thorough understanding of various concepts. However, those skilled in the art will readily recognize that these concepts may be practiced without these specific details. In some instances, this description provides well known structures and components in block diagram form in order to avoid obscuring such concepts.

While this description describes aspects and embodiments by illustration to some examples, those skilled in the art will understand that additional implementations and use cases may come about in many different arrangements and scenarios. Innovations described herein may be implemented across many differing platform types, devices, systems, shapes, sizes, packaging arrangements. For example, embodiments and/or uses may come about via integrated chip embodiments and other non-module-component based devices (e.g., end-user devices, vehicles, communication devices, computing devices, industrial equipment, retail/purchasing devices, medical devices, AI-enabled devices, etc.). While some examples may or may not be specifically directed to use cases or applications, a wide assortment of applicability of described innovations may occur. Implementations may range a spectrum from chip-level or modular components to non-modular, non-chip-level implementations and further to aggregate, distributed, or OEM devices or systems incorporating one or more aspects of the described innovations. In some practical settings, devices incorporating described aspects and features may also necessarily include additional components and features for implementation and practice of claimed and described embodiments.

### Overview

In the present disclosure, the use of limb ballistocardiogram (BCG) measurements for use as an unobtrusive estimation of cardiovascular (CV) parameters is described. The inventors have discovered that a BCG reading from a limb, using the techniques and systems described herein, can be leveraged to generate accurate readings of CV parameters.

For example, during some experiments, certain reference CV parameters were measured (which may include some or all of diastolic, pulse, and systolic pressures, stroke volume, cardiac output, and total peripheral resistance) while an upper-limb BCG reading was taken using an accelerometer embedded in a wearable armband and a whole-body BCG reading was taken based on a strain gauge embedded in a weighing scale, while simultaneously a finger photoplethysmogram (PPG) was acquired. To standardize the analysis, the more convenient yet unconventional armband BCG signal was transformed into the more conventional whole-body BCG (called the synthetic whole-body BCG) using a signal processing procedure. (However, in other embodiments, the armband BCG signal may be utilized without conversion to a synthetic whole-body BCG.) Characteristic features were extracted from these BCG and PPG waveforms, in the form of wave-to-wave time intervals, wave amplitudes, and wave-to-wave amplitudes. Then, the relationship between the characteristic features associated with (i) the whole-body BCG-PPG pair and (ii) the synthetic whole-body-PPG pair versus the CV parameters were analyzed using a machine learning analysis such as multivariate linear regression analysis. The results indicated that each of the CV parameters of interest may be accurately estimated by a combination of as few as two characteristic features in the upper-limb or lower-limb (i.e., whole-body) BCG, and also that the characteristic features recruited for the CV parameters were to a large extent relevant according to the physiological mechanism underlying the BCG.

Further details, systems, techniques, algorithms, and variations thereof will now be described, beyond the foregoing example experiment.

### 1. Ballistocardiogram

The ballistocardiogram (BCG) is the recording of the body movement (including displacement, velocity, and acceleration) in response to the ejection of the blood by the heart. In the absence of any external force acting on the body, the center of mass of the body must remain unchanged. Hence, as the blood circulates in the body, the rest of the body moves in the opposite direction to the circulating blood so that the center of mass of the entire body is maintained. This body movement may be recorded using a wide range of BCG instruments, such as a force plate, weighing scale, bed, and chair. Being a response to the circulation of the blood, the BCG may be closely associated with the CV functions and thus possess clinical value. In fact, the BCG is primarily attributed to the interaction of BP pulses at the aortic inlet and outlet as well as the apex of the aortic arch. Hence, the BCG waveform is largely shaped by the aortic BP waveforms and may thus serve as a window through which the shape of the aortic BP waveforms can be inferred (at least to a certain extent).

Considering that the shape of the BCG may originate from the aortic BP waveforms, it is quite reasonable to conceive that the BCG (especially the characteristic features therein) may have close relationship to the CV parameters. Combined with the unobtrusiveness of the BCG instrumentation, such a capability may open up unprecedented possibilities for ultra-convenient estimation of CV parameters in daily life.

Motivated by this opportunity, the potential of the limb BCG for unobtrusive estimation of CV parameters is herein investigated. The BCG in the head-to-foot direction may be instrumented at the upper limb site using an accelerometer embedded in a wearable armband and at the lower limb sites using a strain gauge embedded in a customized weighing scale, respectively, simultaneously with a finger PPG. The whole-body BCG measured with a weighing scale may be to a large extent analogous to the traditional whole-body BCG measured with a bed. In contrast, there is relatively sparse prior work on the armband BCG. Hence, the whole-body BCG and the armband BCG may present contrasting trade-off between the accuracy of CV parameter estimation and amenity to wearable implementation. To standardize the analysis of these distinct BCG signals, the armband BCG may be transformed into the whole-body BCG (called the synthetic whole-body BCG) using a signal processing procedure. The characteristic features may be extracted from these BCG and PPG waveforms in the form of wave-to-wave time intervals, wave amplitudes and wave-to-wave amplitudes as well as BCG-PPG pulse transit time (PTT; the time interval between a major wave (e.g. a peak or trough) in the BCG waveform and the next diastolic foot/trough of the corresponding PPG waveform). Then, the relationship between the characteristic features associated with (i) the whole-body BCG-PPG pair (e.g., the time series data of a BCG and PPG taken at the same time) and (ii) the synthetic whole-body BCG-PPG pair (e.g., synthetic BCG data aligned in time series with PPG data) versus the CV parameters (including DP, pulse BP (PP), and SP, SV, CO, and TPR) can be analyzed using machine learning techniques such as multivariate linear regression analysis.

### 2. Materials and Methods

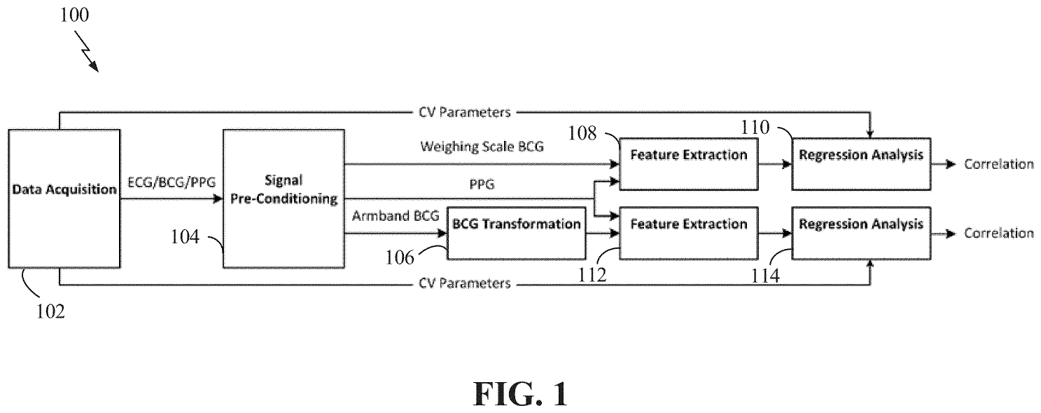

FIG. 1 illustrates a block diagram illustrating a BCG analysis procedure 100 to investigate the potential of limb BCG for CV parameter estimation according to some embodiments. To investigate the potential of the limb BCG for CV parameter estimation, the BCG signals were analyzed with the following procedure 100: (i) experimental data acquisition 102; (ii) signal pre-conditioning 104; (iii) signal processing to transform the armband BCG into the whole-body BCG 106; (iv) feature extraction 108, 112; and (v) machine learning analysis (e.g., regression analysis) 110, 114.

### 2.1. Experimental Protocol

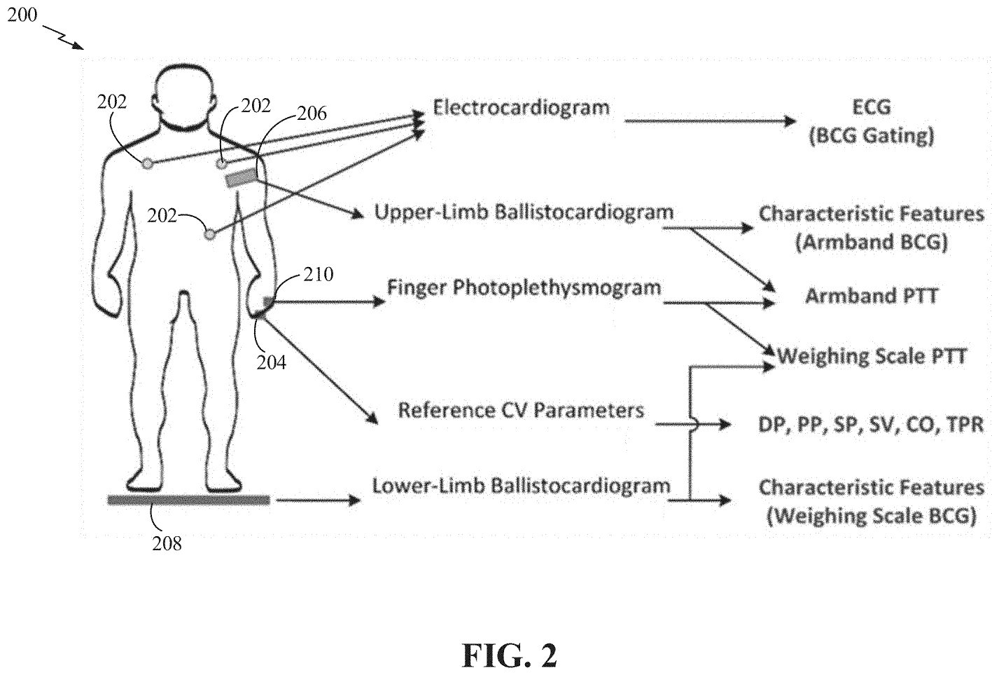

FIG. 2 illustrates instrumented physiological signals according to some embodiments. The data and procedures were verified by human subject study conducted in 17 young healthy volunteers (age 25+/−5 years old; gender 12 male and 5 female; height 174+/−10 cm; weight 74+/−17 kg). From each subject, a wide variety of physiological signal waveforms required for investigating the relationship between the upper-limb (i.e., arm) and lower-limb (i.e., leg) BCG and the CV parameters may be instrumented using sensors as follows. First, the electrocardiogram (ECG) may be instrumented using a number of gel electrodes 202 in a modified Lead II configuration interfaced to a wireless amplifier. In one embodiment, the electrode lead 202 may be BN-EL50 (Biopac Systems, Goleta, Calif., USA). However, it should be appreciated that the electrode lead 202 is not limited to BN-EL50. It could be any other electrode lead which measure and produce an ECG signal. Second, the reference CV parameters (including the BP waveform, SV, CO, and TPR) were instrumented using a fast servo-controlled finger cuff embedded with a blood volume waveform sensor 204 on the ring finger of a hand to implement the volume clamping method. The finger cuff with the blood volume waveform sensor 204 may be ccNexfin System (Edwards Lifesciences, Irvine, Calif., USA). However, it should be appreciated that the finger cuff embedded with the blood volume waveform sensor 204 on the ring finger is not limited to ccNexfin System. It could be any other device, module, or system which measure and produce the reference CV parameters. Third, the upper-limb BCG (called hereafter the armband BCG) was instrumented using a high-resolution accelerometer 206 embedded in an armband equipped with a wireless amplifier. The accelerometer 206 may be BN-ACCL3 (Biopac Systems, Goleta, Calif., USA). However, it should be appreciated that the accelerometer 206 is not limited to BN-ACCL3. It could be any other device, module, or system which measure and produce a BCG signal. Also, the accelerometer is not limited to be embedded in an armband. It could be a wrist BCG instrumented using an accelerometer embedded in a wristband equipped with a wireless amplifier. The accelerometer 206 may be placed any place in an upper limb to produce a BCG signal. Fourth, the lower-limb BCG (called hereafter the whole-body BCG) was instrumented using a strain gauge embedded in a customized weighing scale 208. The weighing scale 208 may be BC534 (Tanita, Tokyo, Japan). However, it should be appreciated that the weighing scale 208 is not limited to BC534. It could be any weighing scale which produces a whole-body BCG signal Fifth, the PPG signal was instrumented using a finger clip sensor 210. The finger clip sensor 210 may be 8000AA (Nonin Medical, Plymouth, Minn., USA). However, it should be appreciated that the sensor 210 is not limited to 8000AA. It could be any suitable sensor which can measure a PPG signal. All the devices were interfaced to a laptop computer by way of a data acquisition unit to synchronously instrument all the waveforms at 1 kHz sampling rate. The data acquisition unit may be MP150 (Biopac Systems, Goleta, Calif., USA). However, it should be appreciated that the data acquisition unit is not limited to MP150. It could be any device, module, or system to synchronously instrument all the waveforms.

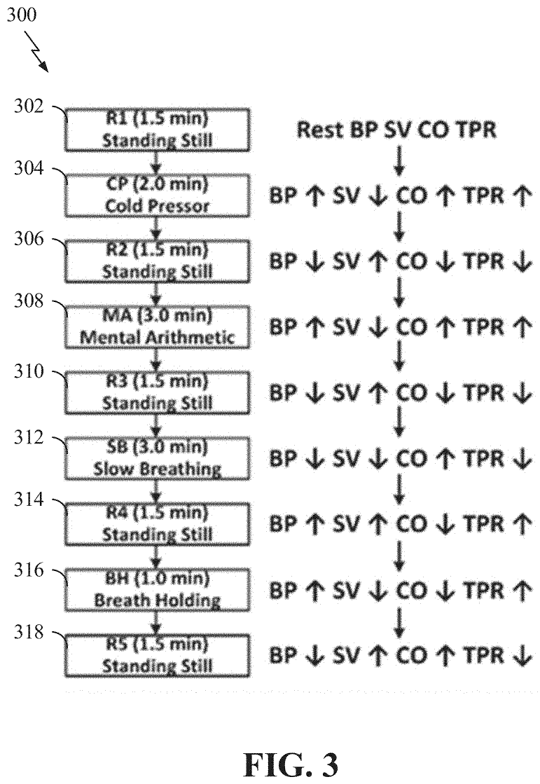

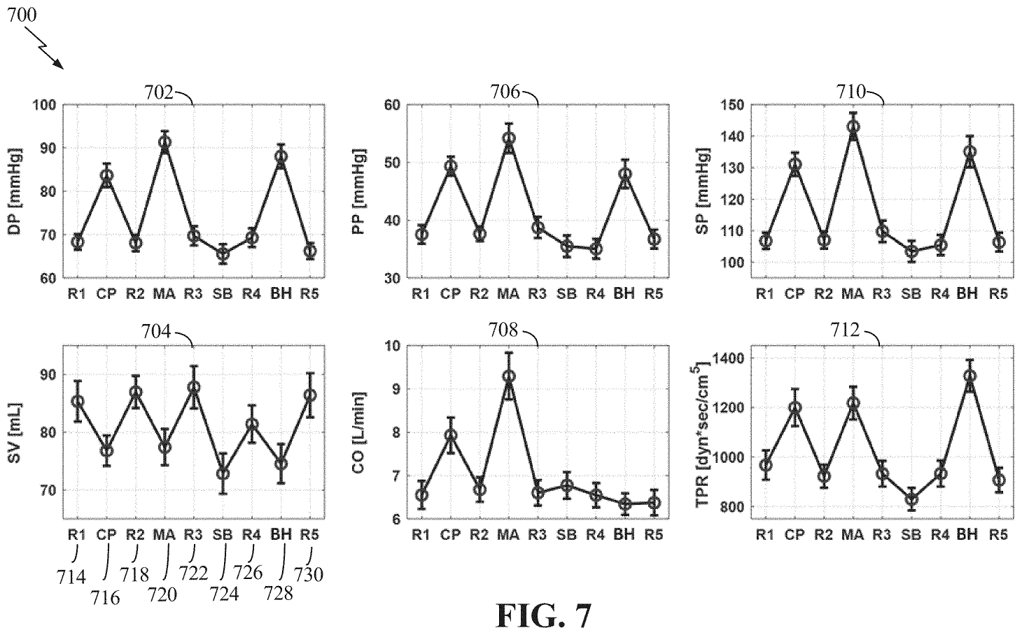

FIG. 3 illustrates a flow chart illustrating hemodynamic interventions. The above-mentioned physiological signal waveforms may be acquired while the subjects undergo four hemodynamic interventions as shown FIG. 3. Each subject may stand still for 1.5 min for an initial rest state (R1) 302. Then, the subject may undergo the cold pressor intervention (CP) 304 for 2 min, in which a free hand of the subject is immersed in ice water. In the CP state, the BP of the subject increases, the SV decreases, the CO increase, and the TPR increases. Followed by standing still for 1.5 min for a second rest state (R2) 306, the subject may undergo the mental arithmetic intervention (MA) 308 for 3 min, the subject repeatedly adds the digits of a three-digit number and added the sum to the original number. After the waveforms are normalized in the R2 state, in the MA state, the BP of the subject increases, the SV decreases, the CO increase, and the TPR increases. Followed by standing still for 1.5 min for a third rest state (R3) 310, the subject may undergo the slow breathing intervention (SB) 312 for 3 min, in which the subject takes deep and slow breaths. After the waveforms are normalized in the R3 state, in the SB state, the BP of the subject decreases, the SV decreases, the CO increase, and the TPR decreases. Followed by standing still for 1.5 min for a fourth rest state (R4) 314, the subject may undergo the breath holding intervention (BH) 316, in which the subject holds breath after normal exhalation. After the waveforms are normalized in the R4 state, in the BH state, the BP of the subject increases, the SV decreases, the CO decreases, and the TPR increases. Lastly, the subject may stand still for 1.5 min for a fifth rest state (R5) 318. The result of the interventions is shown in FIG. 7 and Section 3.1 below in detail. During the interventions, the subjects may stand on the customized weighing scale with their arms placed at the side and still, and their movements minimized. The signal acquisition was continuously made throughout these states. It should be appreciated that the states, the time period for each state, and the order of the states described above are a mere example. The states, the time period for each state, and the order of the states may be other states, a different time period for each state, and a different order of the states if exemplary physiological signal waveforms are acquired.

### 2.2. Signal Pre-Conditioning

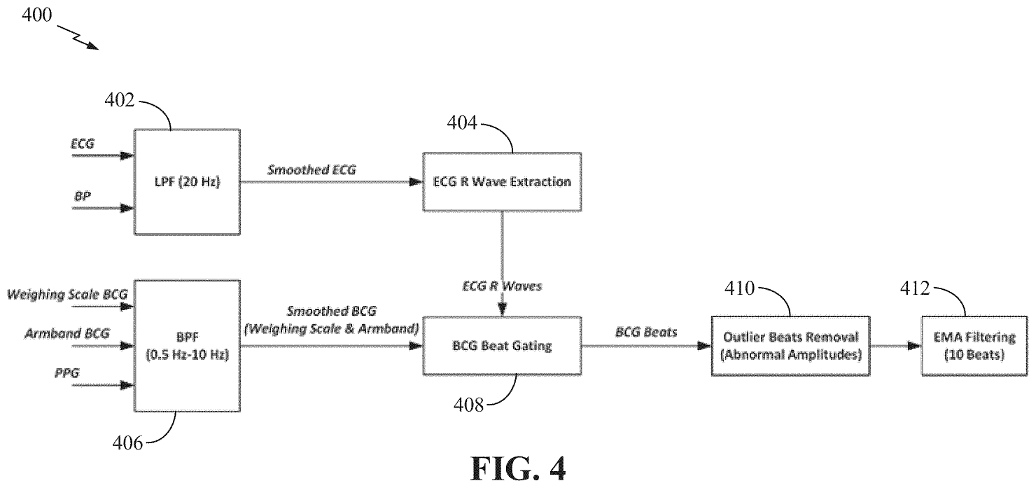

FIG. 4 is block diagram conceptually illustrating a signal preconditioning procedure. In each subject, the acquired data may be segmented into nine periods: R1, CP, R2, MA, R3, SB, R4, BH, and R5. Then, the physiological signal waveforms may be pre-conditioned as follows on a period-by-period basis. First, the signals may be smoothed via zero-phase filtering: the ECG and BP by a low-pass filter 402. The low-pass filter 402 may be a 1st-order Butterworth low-pass filter with a predetermined cut-off frequency. The cut-off frequency may be 20 Hz. However, it should be appreciated that the low-pass filter 402 is not limited to the 1st-order Butterworth low-pass filter, and the predetermined cut-off frequency is not limited to 20 Hz. The type of low-pass filter may be any suitable low-pass filter to smooth the ECG and BP by zero-phase filtering the ECG and BP. The BCG and PPG may be smoothed by a band-pass filter 406. The band-pass filter 406 may be a 2^{nd}-order Butterworth band-pass filter with a predetermined pass band. The predetermined pass band may be 0.5˜10 Hz. However, it should be appreciated that the band-pass filter could be any other type of band-pass filter which smooths the BCG and PPG. Further, the predetermined pass band may be other frequency range for smoothing the BCG and PPG.

Second, the ECG R wave may be extracted using the Pan Tompkins method 404. However, it should be appreciated that the ECG R wave may be extracted any other method or filter. Third, the BCG and PPG beats may be gated with predetermined time instants before the R wave as gating locations 408. The predetermined time instants may correspond to 10% of cardiac period. Fourth, beats associated with the low-quality armband and/or whole-body BCG waveforms may be discarded 410. One example to discard the low-quality armband and/or whole-body BCG waveforms is to (i) calculate the amplitudes associated with all the armband and/or whole-body BCG beats, and (ii) discard the beats associated with extraordinarily large or small BCG amplitude (i.e., outside of 3 scaled median absolute deviations (with the scaling factor of 1.4826) around the median amplitude). Fourth, the armband and whole-body BCG signals were smoothed using an exponential moving average filter 412 to suppress the adverse impact of motion artifacts. The filter 412 may be a 10-beat exponential moving average filter. However, the filter 412 may be any other suitable filter which suppresses the adverse impact of motion artifacts.

### 2.3. Analysis of Whole-Body BCG for CV Parameter Estimation

The whole-body BCG may be analyzed to investigate its association with the CV parameters in the following steps: (i) feature extraction and (ii) machine learning analysis (e.g., multivariate regression analysis).

### 2.3.1. Feature Extraction

The whole-body BCG may be labeled for the major I, J, and K waves as follows. The J wave may be determined by finding the maximum peak in each BCG beat appearing after the ECG R wave. Then, the I and K waves may be determined by finding the local minima right before and after the J wave, respectively. The foot of the PPG may be determined using the intersecting tangent method. By using these labels, a total of 16 characteristics features listed in Table 1 was constructed.

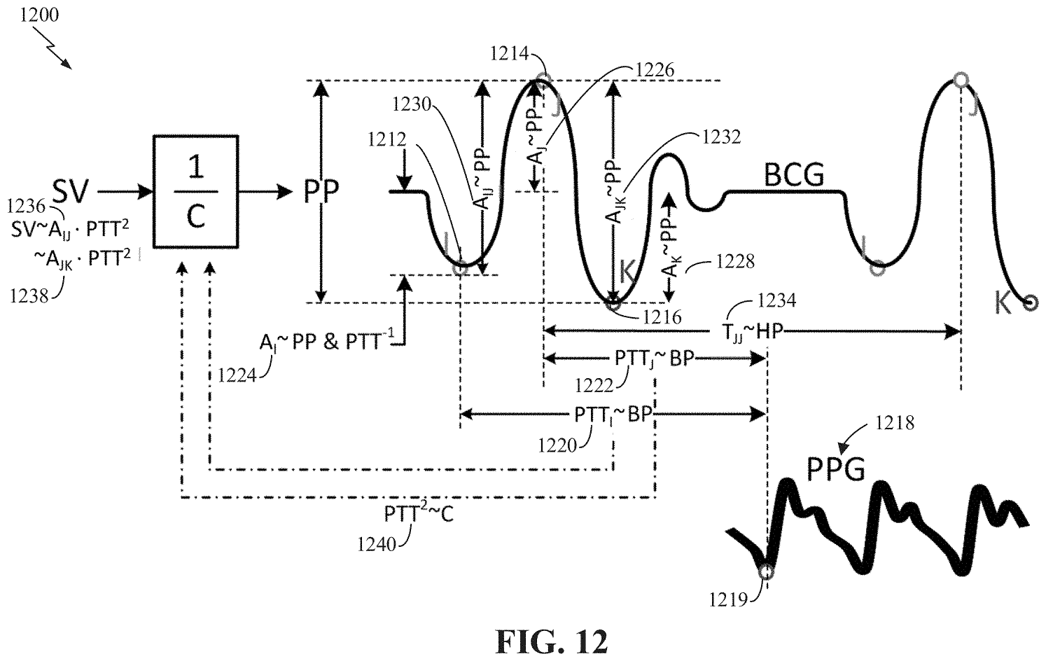

TABLE 1
Characteristic features extracted from the ballistocardiogram (BCG)
in conjunction with the photoplethy smogram (PPG) shown in FIG. 12.

| Symbol | Definition |
|---|---|
| PTT_{I} (1220) | Time interval between BCG I wave and PPG foot |
| PTT_{J} (1222) | Time interval between BCG J wave and PPG foot |
| PTT_{K} | Time interval between BCG K wave and PPG foot |
| T_{IJ} | Time interval between BCG I wave and J wave |
| T_{JK} | Time interval between BCG J wave and K wave |
| T_{IK} | Time interval between BCG I wave and K wave |
| T_{JJ} | Time interval between J waves of two consecutive BCG beats |
| A_{I} | Amplitude of BCG I wave |
| A_{J} (1226) | Amplitude of BCG J wave |
| A_{K} (1228) | Amplitude of BCG K wave |
| A_{IJ} (1230) | Amplitude difference between I wave and J wave |
| A_{JK} (1232) | Amplitude difference between J wave and K wave |
| A_{IJ} · PTT_{I}² (1236) | Surrogate of SV* |
| A_{JK} · PTT_{I}² (1238) | Surrogate of SV* |
| RMS | Root mean square of BCG waveform B[i], i = 1~N: |
| | Σᵢ₌₁ⁿ \sqrt{B[i]²/n} |
| E | Energy of BCG waveform B[i], i = 1~N: Σᵢ₌₁ⁿ B[i]² |

*Considering that A_{IJ} and A_{JK} are approximately associated with PP and PTT² is proportional to arterial compliance, A_{IJ} · PTT_{I}² and A_{JK} · PTT_{I}² are approximately associated with SV.

### 2.3.2. Data Analysis

The data may be analyzed in the following steps. First, the outliers in the extracted characteristic features may be identified and removed. Second, the sample size of the characteristic features may be increased. Third, the relationship between the characteristic features and the CV parameters may be analyzed. The analysis may be performed on the subject-by-subject basis.

First, the outliers in the characteristic features may be extracted from the BCG and PPG signals as follows. In each of the nine rest and intervention periods associated with each subject, the time series sequences of the characteristic features are examined. Each 3 consecutive samples in the time series are inspected for possible outliers in a 9-sample window (including 3 samples before and 3 samples after the inspected samples). An outlier may be identified if a sample was outside of 3 scaled median absolute deviations around the median of the 9 characteristic feature samples. If >75% of the beats are removed, the period itself may be excluded from subsequent analysis. Subjects in which <6 rest and intervention periods are available for analysis may be also excluded from subsequent analysis.

Second, the sample size of the characteristic features may be increased using the bootstrap technique similarly to prior work so as to conduct robust machine learning analysis including regression analysis (i.e., to reliably determine the coefficients in the regression models). More specifically, in each of the nine rest and intervention period associated with each subject, the time intervals at which the CV parameters and the characteristic features attained stable extrema may be determined (see Table 2 for the definition of the extrema). Then, 11 samples in the vicinity of the extrema are taken, the average of which are used as the representative CV parameter and characteristic feature values. In addition, each of the CV parameters and characteristic features may be approximated as the corresponding parametric bootstrap based on the mean and standard variation of the 11 samples. Then, 100 bootstrap samples may be created using the Monte Carlo method. Each bootstrap sample may be created by (i) creating 11 random Monte Carlo samples and (ii) taking their average. Hence, up to 900 bootstrap samples (corresponding to the nine rest and intervention periods) may be created in each subject. In each subject, the bootstrap samples of CV parameters and characteristics features associated with all the rest and intervention periods may be merged for machine learning analysis (e.g., multivariate regression analysis).

TABLE 2
Extremum regions of cardiovascular (CV) parameters
in individual rest and intervention periods.

| | R1 | CP | R2 | MA | R3 | SB | R4 | BH | R5 |
|---|---|---|---|---|---|---|---|---|---|
| DP | Min | Max | Min | Max | Min | Min | Min | Max | Min |
| PP | Min | Max | Min | Max | Min | Min | Min | Max | Min |
| SP | Min | Max | Min | Max | Min | Min | Min | Max | Min |
| SV | Max | Min | Max | Min | Max | Min | Max | Min | Max |
| CO | Min | Max | Min | Max | Min | Min | Min | Max | Min |
| TPR | Min | Max | Min | Max | Min | Min | Min | Max | Min |

Third, machine learning analysis (e.g., multivariate linear regression analysis) may be conducted at the individual subject level, or alternatively, in a group of many subjects, to investigate the potential of the whole-body BCG for unobtrusive estimation of CV parameters. First, machine learning models (e.g., multivariate linear regression models) associated with each of the CV parameters may be developed using the bootstrap samples. Then, the validity of these models may be tested using the representative CV parameters and characteristic features at the extrema associated with all the available rest and intervention periods of the subject (≤9; FIG. 3). The goal of the machine learning analysis (e.g., multivariate regression analysis) is to determine (i) the most predictive characteristic features for the CV parameters as well as (ii) the number of characteristic features required to achieve high degree of correlation (r≥0.7) with the individual CV parameters for accurate unobtrusive estimation. Hence, all possible combinations of the characteristics features are considered exhaustively, and the models exhibiting high degree of correlation and equipped with physiologically relevant characteristic features are selected. The Pearson's correlation coefficient may be used for determining the univariate characteristics features closely correlated with the CV parameters as well as for assessing the performance of the machine learning models (e.g., multivariate linear regression models).

### 2.4. Analysis of Armband BCG for CV Parameter Estimation

The armband BCG may be analyzed to investigate its association with the CV parameters in the following steps: (i) transformation of the armband BCG to the whole-body BCG, (ii) feature extraction, and (iii) machine learning analysis (e.g., multivariate regression analysis).

### 2.4.1. Transformation of Armband BCG to Whole-Body BCG

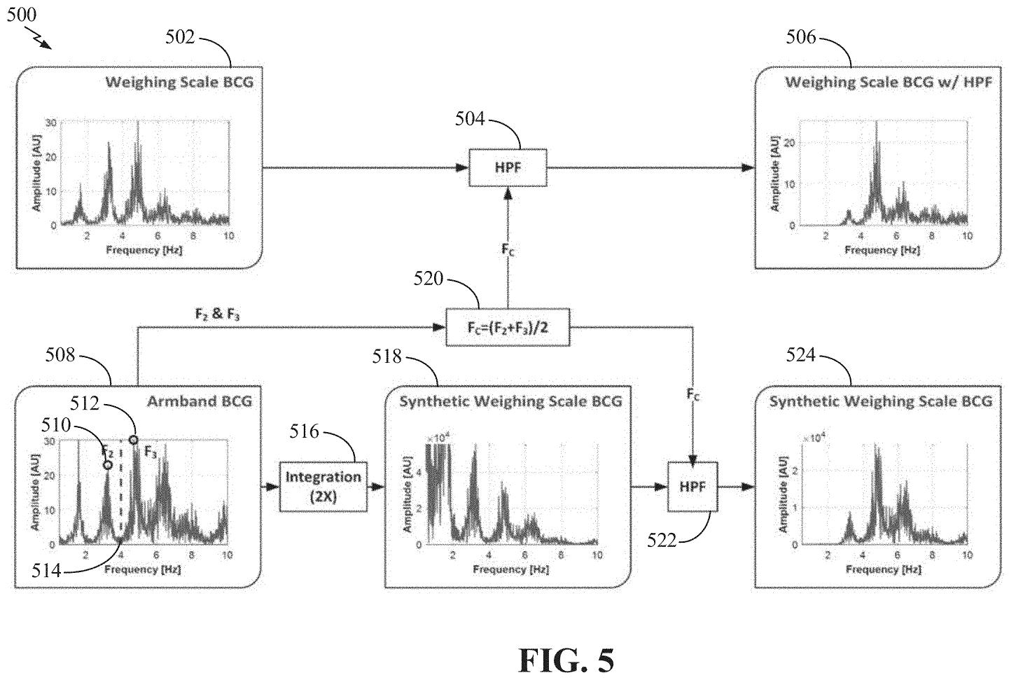

The armband BCG and the whole-body BCG are distinct in waveform morphology due to the difference in the measurement modality involved: the former is an acceleration measurement whereas the latter is a displacement measurement. The relationship between the upper-limb acceleration BCG and CV parameters may be obscure due to the compliance of the body compared with the lower-limb displacement BCG. Hence, the armband BCG may be transformed into an equivalent whole-body BCG. Given that the primary source of the discrepancy between the armband BCG and the whole-body BCG is the measurement modality (i.e., accelerometer versus strain gauge) if the body is assumed to be rigid, this may be accomplished by applying two integrations to the armband BCG as shown in FIG. 5.

FIG. 5 is a block diagram conceptually illustrating a procedure for transforming armband BCG to whole-body BCG according to some embodiments. More specifically, the armband BCG 508 may be integrated in time twice 516 to yield the synthetic whole-body BCG. The integration may use the trapezoidal method. However, it should be appreciated that the integration method is not limited to the trapezoidal method. It may be any other suitable integration method. Then, the synthetic whole-body BCG may be zero-phase filtered using a high-pass filter 522 to remove the low-frequency drift therein. The high-pass filter may be a 4^{th}-order Butterworth high-pass filter. However, the high-pass filter may be any other suitable high-pass filter which removes the low-frequency drift in the synthetic whole-body BCG signal. The cut-off frequency 514 of the filter was determined so that the power spectra (especially in terms of the primary spectral peaks) associated with the whole-body BCG and the synthetic whole-body BCG were made consistent. The comparison of the power spectra associated with the whole-body BCG 506 and the synthetic whole-body BCG 524 may show that the latter exhibited largely higher spectra up to the 2^{nd} spectral peak compared to the former. Hence, the cut-off frequency 514 may be determined empirically as the average of the 2^{nd} and 3^{rd} peaks 510, 512 in the BCG power spectrum 520. Practically, the cut-off frequency can be easily computed from the heart rate as 2.5 times the heart rate, since the spectral peaks in the BCG represent the heart rate and its harmonics. The above-described procedure may be performed in each subject on a period-by-period basis.

The beat-by-beat quality of the whole-body BCG and synthetic weighing BCG calculated from arm BCG may be quantitatively assessed via the following criteria: (1) ∥s[i]−s̅∥/∥s̅−m∥>1, where s[i] is individual BCG beat in an individual (rest or intervention) period, s̅ is the ensemble average of all beats in the individual period, m is the mean of s̅; (2) correlation coefficient between s[i] and s̅ less than 0.5 in each period; (3) a peak with a prominence of >0.25 is detected from the 2^{rd} derivative of the BCG waveform from I wave to K wave as a measure of distortion in the BCG waveform. All beats not fulfilling these criteria may be removed from further analysis.

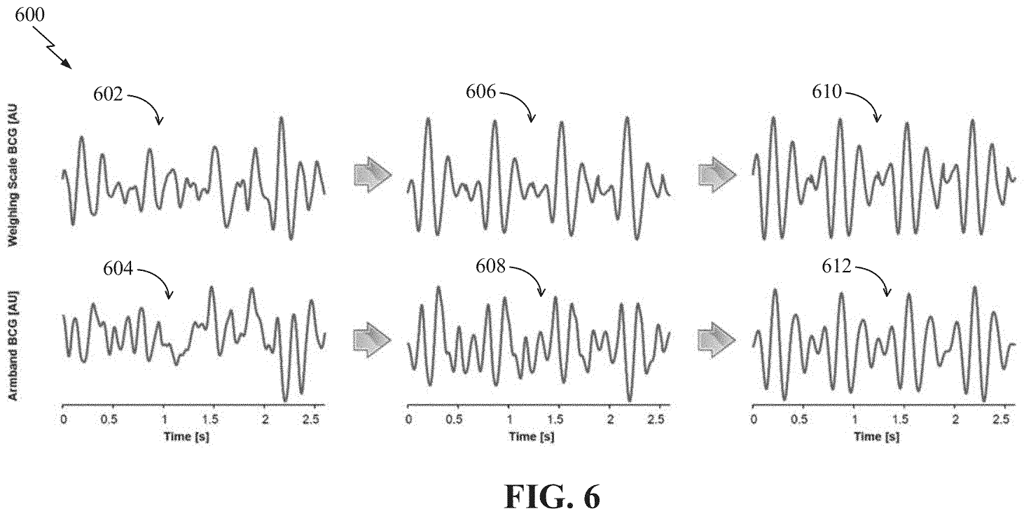

FIG. 6 illustrates representative whole-body and armband BCG waveforms according to some embodiments. Raw whole-body BCG signal 602 and raw armband BCG signal 604 may undergo the signal pre-conditioning procedure. After the signal pre-conditioning procedure, the raw signals 602, 604 may be modulated using an exponential moving average (EMA) filter. The signals 606, 608 after EMA filtering are shown in the middle panel of FIG. 6. The armband BCG signal may be transformed to the synthetic whole-body signal 612.

### 2.5. Feature Extraction and Data Analysis

Feature extraction and data analysis were conducted in the same way as the whole-body BCG, as described in detail in Section 2.3.

### 3. Results

### 3.1. Feature Extraction and Data Analysis

FIG. 7 shows the trends of the changes in the CV parameters in response to the hemodynamic interventions illustrated in FIG. 3. DP 702, PP 706, SP 710, and TPR 712 increase in response to CP 716, MA 720, and BH 728, while decrease in response to SB 724. CO 708 likewise increases in response to CP 716 and MA 720. But, it modestly increases in response to SB 724 and modestly decreases in response to BH 728. SV 704 decreases in response to all the hemodynamic interventions. Noting that CO 708 increases in CP 716 and MA 720, the decrease in SV 704 may be attributed to a large increase in heart rate which shortens the left ventricular ejection time yet still increases CO 708. On the other hand, the decrease in SV 704 in SB 724 and BH 728 may be associated with the marginal change in CO 708 and decrease in heart rate, which is anticipated from the findings of prior studies. These trends may be used in defining the extrema associated with the CV parameters in Table 2.

3.2. CV Parameter Estimation with Whole-Body BCG

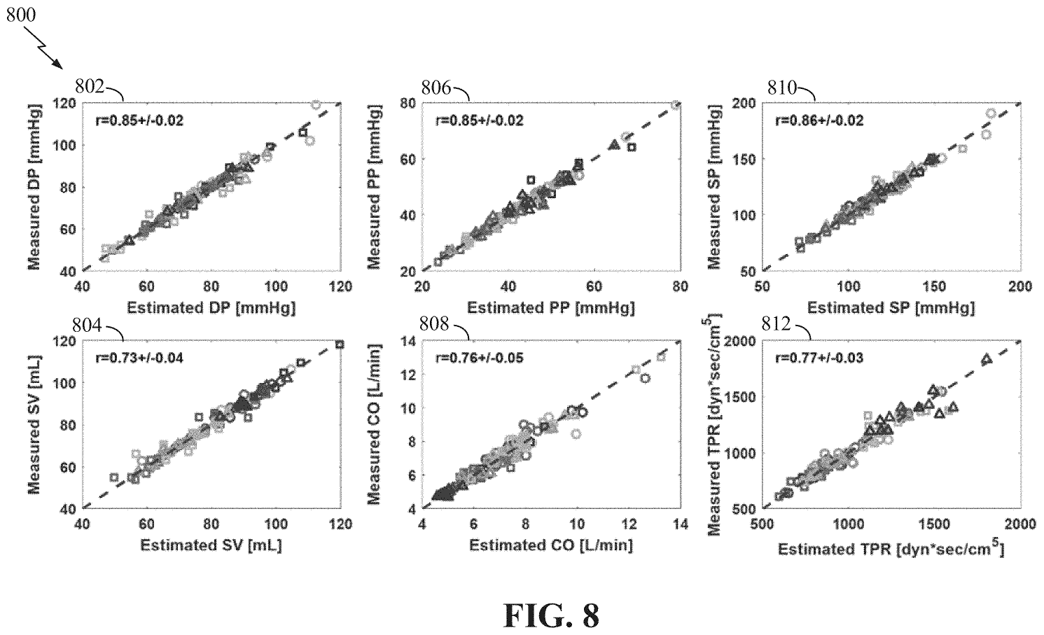

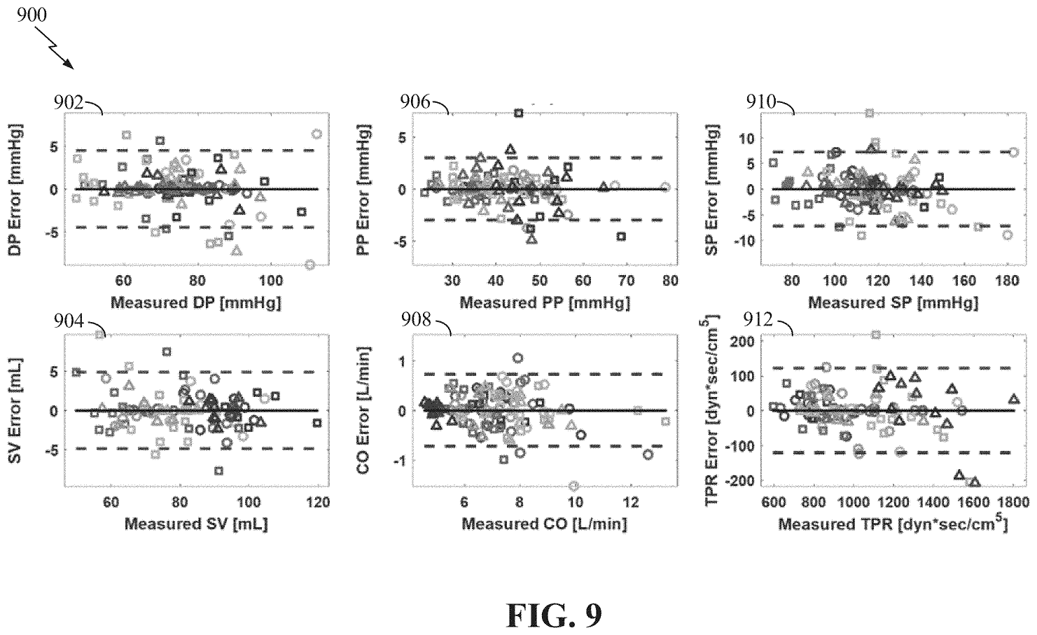

The number of subjects available for machine learning analysis (e.g., multivariate linear regression analysis) after the outlier removal (i.e., subjects with ≥6 rest and intervention periods available for analysis; see Section 2.5 for details) is ≥14 for all the CV parameters associated with the whole-body BCG. Machine learning analysis (e.g., multivariate linear regression analysis) suggests that each of the CV parameters of interest may be accurately estimated by a combination of as few as two characteristic features. In contrast, the best correlation coefficients achieved by univariate characteristic features may be on the average high for DP (0.81) and SP (0.82) but not sufficiently high for the remaining CV parameters (<0.65). For the whole-body BCG at the univariate level, DP may be correlated well with PTT_{I} (r=−0.81+/−0.02) and PTT (r=−0.69+/−0.04), PP may be correlated reasonably with PTT_{I} (r=−0.65+/−0.05) and PTT (r=−0.57+/−0.07) as well as A_{JK} (0.54+/−0.07) and A_{IJ} (0.53+/−0.07), and SP may be correlated well with PTT_{J} (r=−0.82+/−0.02) and PTT (r=−0.72+/−0.04). SV may be correlated most strongly with A_{J} (r=0.50+/−0.09). CO may be correlated with T_{JJ} (r=−0.57+/−0.11), and to a lesser extent, with PTT_{I} and PTT_{J}. TPR may be likewise correlated with PTT_{I} (r=−0.58+/−0.07) and PTT_{J} (r=−0.52+/−0.09) but also with T_{JJ} (r=0.54+/−0.07). Table 3 may show the best-performing (a) univariate and (c) bivariate regression models associated with the whole-body BCG. FIGS. 8-9 may show (a) the correlation plot (FIG. 8) and (b) the Bland-Altman plot between measured versus regressed CV parameters associated with the whole-body BCG (FIG. 9). The dashed lines in FIGS. 8-9 indicate confidence intervals while horizontal solid lines in FIG. 9 indicates bias. FIG. 12 summarizes the relationship between the characteristic features in the whole-body BCG and the CV parameters (Section 4.2).

3.3. CV Parameter Estimation with Armband BCG

The signal processing procedure (FIGS. 4-6) may drastically improve the correlation between the measured versus synthetic whole-body BCG compared to the correlation between the measured whole-body versus armband BCG, both at all the individual rest and intervention states as well as across all the rest and intervention states (r=0.70 versus r=0.52 on the average).

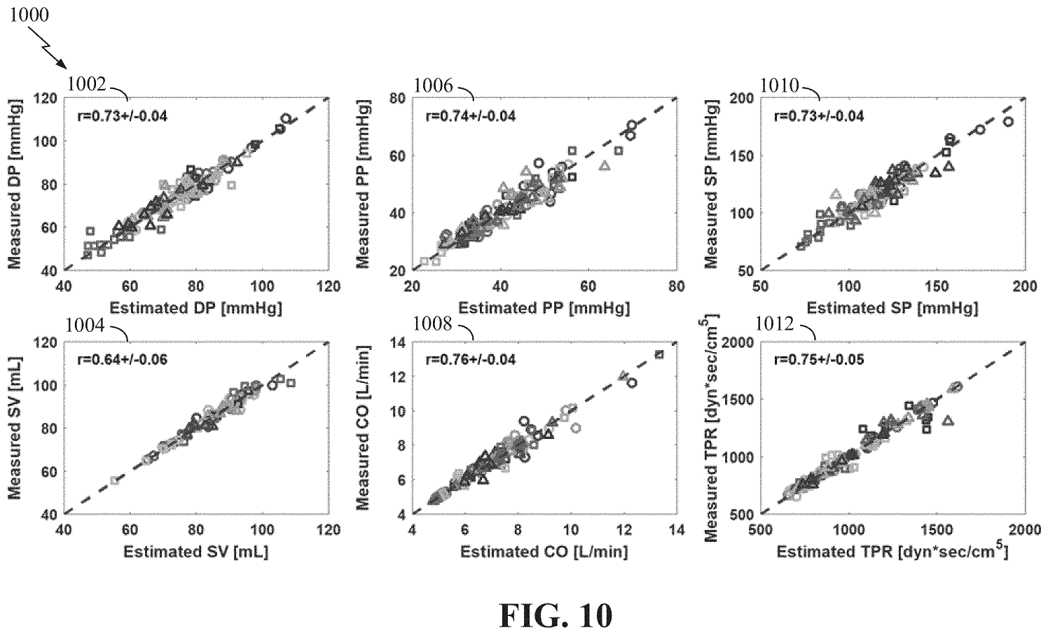

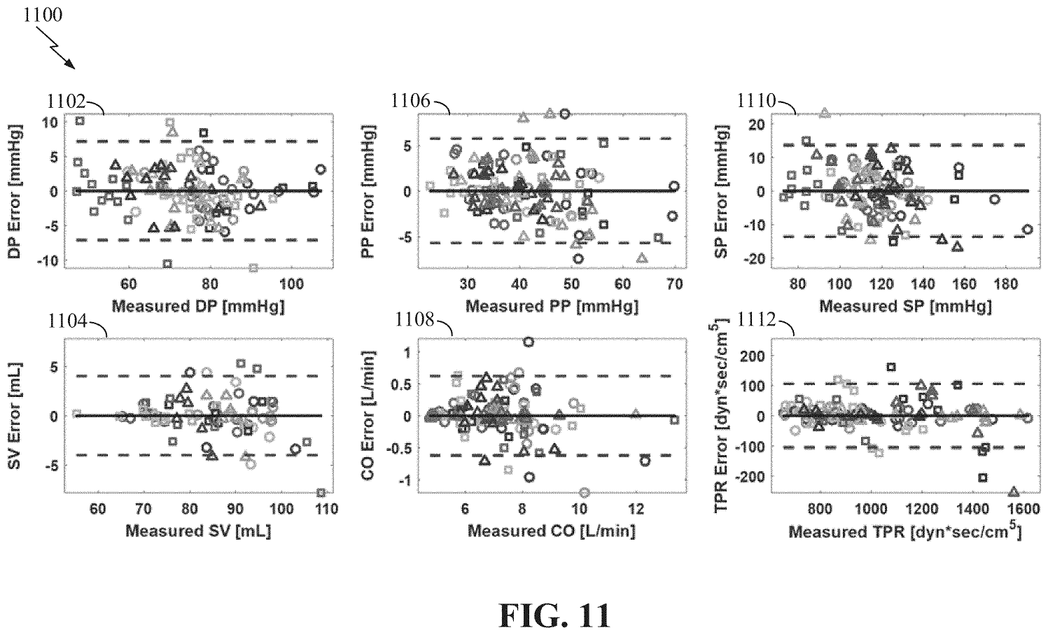

The number of subjects available for machine learning analysis (e.g., multivariate linear regression analysis) after the outlier removal is ≥14 for all the CV parameters associated with the armband BCG except SV (12 subjects). Machine learning analysis (e.g., multivariate linear regression analysis) suggests that each of the CV parameters of interest may be accurately estimated by a combination of as few as two characteristic features in the upper-limb BCG. In contrast, the best correlation coefficients achieved by univariate characteristic features may be in general low (<0.57) for all CV parameters. For the armband BCG at the univariate level, DP 1002 may be correlated with PTT (r=−0.36+/−0.12) and PTT_{I} (r=−0.34+/−0.15), PP 1006 may be correlated with PTT (r=−0.53+/−0.06) and PTT_{I} (r=−0.48+/−0.08), and SP 1010 may be correlated with PTT (r=−0.42+/−0.11). SV 1004, CO 1008, and TPR 1012 may be most strongly correlated with T_{JJ} (r=0.34+/−0.10, −0.57+/−0.10, and 0.50+/−0.10). Table 3 shows the best-performing (b) univariate and (d) bivariate regression models associated with the synthetic whole-body BCG. FIGS. 10-11 show (a) the correlation plot (FIG. 10) and (b) the Bland-Altman plot between measured versus regressed CV parameters associated with the synthetic whole-body BCG (FIG. 11). FIG. 12 summarizes the relationship between the characteristic features in the armband BCG and the CV parameters.

TABLE 3
Representative univariate and bivariate regression models with physiological interpretability associated
with whole-body ballistocardiogram (BCG) and synthetic whole-body BCG transformed from armband BCG.

| | | | | | |
|---|---|---|---|---|---|
| DP | PP | SP | SV | CO | TPR |

(a) Whole-body BCG (r: mean +/− SE)

| | | | | | | |
|---|---|---|---|---|---|---|
| Features | PTT_{I} | PTT_{I} | PTT_{I} | A_{J} | T_{JJ} | PTT_{I} |
| r | 0.81 +/− 0.02 | 0.65 +/− 0.05 | 0.82 +/− 0.02 | 0.50 +/− 0.09 | 0.57 +/− 0.11 | 0.58 +/− 0.07 |

(b) Synthetic whole-body BCG (r: mean +/− SE)

| | | | | | | |
|---|---|---|---|---|---|---|
| Features | PTT_{J} | PTT_{J} | PTT_{J} | T_{JJ} | T_{JJ} | T_{JJ} |
| r | 0.36 +/− 0.12 | 0.53 +/− 0.06 | 0.42 +/− 0.11 | 0.34 +/− 0.10 | 0.57 +/− 0.10 | 0.50 +/− 0.10 |

(c) Whole-bodv BCG (r: mean +/− SE)

| | | | | | | |
|---|---|---|---|---|---|---|
| Features | PTT_{I}, A_{I} | PTT_{I}, A_{IJ} | PTT_{I}, A_{JK} | A_{J}, A_{JK} | T_{JJ}, PTT_{J} | T_{JJ}, A_{IJ} · PTT_{I}² |
| r | 0.85 +/− 0.02 | 0.85 +/− 0.02 | 0.86 +/− 0.02 | 0.73 +/− 0.04 | 0.76 +/− 0.05 | 0.77 +/− 0.03 |

(d) Synthetic whole-bodv BCG (r: mean +/− SE)

| | | | | | | |
|---|---|---|---|---|---|---|
| Features | PTT_{I}, A_{I} | T_{JJ}, PTT_{I} | A_{JK}, A_{IJ} · PTT_{I}² | T_{JJ}, A_{IJ} · PTT_{I}² | T_{JJ}, PTT_{J} | T_{JJ}, A_{JK} · PTT_{I}² |
| r | 0.73 +/− 0.04 | 0.74 +/− 0.04 | 0.73 +/− 0.04 | 0.64 +/− 0.06 | 0.76 +/− 0.04 | 0.75 +/− 0.05 |

### 4. Discussion

Direct measurement of the CV parameters necessitates inconvenient and costly equipment and procedures as well as trained operators. The BCG is closely associated with the aortic BP. Considering the pulse contour techniques in deriving the CV parameters from arterial BP waveforms, the BCG may have potential value in estimating the CV parameters. Yet, prior work to investigate the feasibility of estimating the CV parameters from the BCG is quite rare. The work in this application rigorously examines, perhaps for the first time, the relationship between the characteristic features in the limb BCG and the CV parameters.

### 4.1. Potential of Scale and Arm BCG in CV Parameter Estimation

The results from the correlation and machine learning regression analysis suggest that the limb BCG may have the potential to enable unobtrusive CV parameter estimation. For the whole-body BCG, the pair of two features could achieve close correlations with CV parameters (r≥0.85 for all BP and r≥0.73 for SV, CO, and TPR on the average; Table 3(a)). For the armband BCG, the pair of two features extracted from the synthetic whole-body BCG which may transform from the armband BCG could likewise achieve close correlations with CV parameters (r≥0.73 for all BP, r≥0.75 for CO and TPR, and r=0.64 for SV on the average; Table 3(b)). In general, the whole-body BCG outperforms the armband BCG. This may be attributed to (i) the more stable measurement setting for the whole-body BCG relative to the armband BCG and (ii) the errors induced by the transformation of the armband BCG to the synthetic whole-body BCG (see Section 4.4 for details). Indeed, the upper limb may be more susceptible to involuntary movement than the lower limb in contact with the weighing scale. Further, the synthetic whole-body BCG transformed from the armband BCG is not exactly identical to the whole-body BCG (which may explain why the features selected for whole-body BCG and synthetic whole-body BCG were not identical in Table 3). These artifacts combined may result in the deterioration in efficacy of the armband BCG relative to the whole-body BCG in estimating the CV parameters. Regardless, the degree of correlation between the armband BCG and the CV parameters is still adequate.

The adequate correlation between the armband BCG and the CV parameters appears to have benefited from the signal processing procedure developed here to transform the armband BCG to whole-body BCG. Considering the distinct waveform morphology associated with the whole-body BCG versus the armband BCG, the efficacy of the signal processing procedure may have a significant implication on the feasibility of standardized analysis of both the BCG. Arguably, the improvement in the correlation between the measured versus synthetic whole-body BCG compared to the correlation between the measured whole-body versus armband BCG may suggest that the armband BCG can now be analyzed in the same way as the whole-body BCG, the analysis method for which is much more established in the sense that the whole-body BCG may approximately represent the whole-body BCG (i.e., the BCG associated with the movement of the main trunk).

### 4.2. Physiological Relevance of Whole-Body BCG Features

The characteristic features in the whole-body BCG exhibiting close correlation with the CV parameters are physiologically relevant as described below (Table 3(a) and (c)). FIG. 12 may illustrate the relationship between characteristic features in the whole-body BCG and CV parameters. As can be seen, the BCG data exhibits a periodic, repeating waveform having peaks and troughs. Likewise, the PPG signal exhibits its own periodic, repeating waveform, having a trough and peaks.

First, physiologically relevant whole-body BCG features may exhibit close correlation to the CV parameters in the univariate regression analysis. The correlation of DP with PTT_{I} 1220 (the time interval between the first trough 1212 of the whole-body BCG signal 1200 and the next foot 1218 of the corresponding PPG signal) and PTT_{J} 1222 (the time interval between the first peak 1214 of the whole-body BCG signal 1200 and the next foot 1219 of the PPG signal 1218) is consistent with the established fact that DP is correlated closely to PTT. The correlation of PP with PTT_{I} 1220 and PTT_{J} 1222 may be understood by the fact that PP may be (at least in a local sense) inversely proportional to PTT. The correlation of PP with A_{JK} 1232 and A_{IJ} 1230 may be understood by the fact that the amplitude features A_{J} 1226 and A_{JK} 1232 may be the surrogates of ascending aortic and descending aortic PP as well as the fact that an increase in PP may lead to an increase in the overall BCG amplitude. The correlation of SP with PTT_{I} 1220 and PTT_{J} 1222 may be understood from the correlation between DP and PTTs in conjunction with the fact that the hemodynamic interventions considered in this work elicited concurrent increase in both DP and SP. Likewise, physiologically relevant whole-body BCG features were properly correlated with SV, CO, and TPR in the univariate regression analysis, though not as strong as BP. The correlation of SV with A_{J} 1226 is reasonable in that A_{J} 1226 may be the surrogate of ascending aortic PP and that SV and PP are proportional to each other if the arterial compliance (C) does not change largely (PP=SV/AC). The correlation of CO with T_{JJ} 1234, and to a lesser extent, with PTT_{I} 1220 and PTT_{J} 1224 may also be reasonable by noting that CO is the product of SV and heart rate, T_{JJ} 1234 is a surrogate of heart rate, and PTT_{I} 1220 and PTT_{J} 1222 are correlated with PP (which is proportional to SV). The negative correlation of TPR and PTT_{I} 1220 and PTT_{J} 1224 appears reasonable given that the changes in BP and TPR are in phase (FIG. 7). In contrast, the positive correlation of TPR with T_{JJ} 1234 is counter-intuitive in that BP, heart rate, and TPR mostly change in the same direction, except in BH (the change in heart rate may be deduced from SV and CO in FIG. 7 as CO/SV). It is speculated that the large inverse change in TPR and heart rate in BH appears to dominate the relatively small in-phase changes in the remaining hemodynamic interventions and thereby yielded the positive correlation between TPR and T_{JJ} 1234. Hence, the positive correlation between TPR and T_{JJ} 1234 as observed in this work may not generalize.

Second, the whole-body BCG features selected in the bivariate regression analysis were also quite physiologically relevant (FIG. 12). DP was regressed with PTT_{I} 1220 and A_{I} 1224 (r=0.85+/−0.02), which is relevant in that A_{I} 1224 may be inversely proportional to BP since a decrease in PTT (corresponding to an increase in BP) may be associated with a decrease in A_{I} 1224. PP was regressed with PTT_{I} 1220 and A_{IJ} 1230 (r=0.85+/−0.02) consistently to the univariate regression analysis. SP was regressed with PTT_{I} 1220 and A_{JK} 1232 (r=0.86+/−0.02), which is relevant in that A_{JK} 1232 may represent PP as mentioned above. SV was regressed with A_{J} 1226 and A_{JK} 1232 (r=0.73+/−0.04), which is supported by the close relationship between these amplitude features and PP and the proportionality between PP and SV under small arterial compliance (C) change. SV was also regressed well with T_{JJ} 1234 and RMS (r=0.73+/−0.04), which may be due to the inversely proportional change between SV and HR (FIG. 7; which may be specific to the data analyzed in our work due to large changes in HR and thus may not generalize) and the proportional association between the amplitude features and RMS. CO was regressed with T_{JJ} 1234 (which is consistent with the univariate regression case) and PTT_{J} 1222 (r=0.76+/−0.05). The correlation between CO and PTT_{J} 1222 appears relevant because CO and SV are proportional, SV and PP may be proportional, and PP is locally inversely proportional to PTT (as stated above). TPR was regressed with T_{JJ} 1234 (consistently to the univariate regression analysis) and A_{IJ}·PTT_{I}² 1236 (r=0.77+/−0.03). Considering that A_{IJ} 1230 may serve as a surrogate of PP (as stated above) and that PTT² 1240 is proportional to arterial compliance according to the wave speed equation, A_{IJ}·PTT_{I}² 1236 may be regarded as a surrogate of SV. Hence, it may qualify for a feature to track the trend of TPR given its inversely proportional relationship to TPR (r=−0.32+/−0.08; FIG. 7).

### 4.3. Physiological Relevance of Armband Scale BCG Features

The characteristic features in the synthetic whole-body BCG transformed from the armband BCG exhibiting close correlation with the CV parameters may be physiologically relevant to a large extent as described below (Table 3(b) and (d)). FIG. 12 may also illustrate the relationship between characteristic features in the armband scale BCG and CV parameters.

First, many physiologically relevant synthetic whole-body BCG features may exhibit correlation to the CV parameters in the univariate regression analysis consistently to the whole-body BCG. However, the degree of correlation was not as strong as the whole-body BCG.

Second, the synthetic whole-body BCG features selected in the bivariate regression analysis may be likewise quite physiologically relevant and largely consistent with the whole-body BCG case (FIG. 12). DP may be regressed with PTT_{I} 1220 and A_{I} 1224 (r=0.73+/−0.04). PP may be regressed with PTT_{I} 1220 and T_{JJ} 1234 (r=0.74+/−0.04). PTT_{I} 1220 may have been selected since it changed in the opposite direction to DP, PP, and SP in this work. T_{JJ} 1234 may have been selected since it exhibits a positive correlation with SV in this work (which may be deduced from SV and CO in FIG. 7). SP may be best regressed with A_{JK} 1232 and A_{IJ}·PTT_{I}² 1236 (r=0.73+/−0.04). This correlation may be understood in that SP and PP mostly change in the same direction in response to the interventions considered in this work (FIG. 7). However, SP may be also well regressed with the pair of PTT_{J} 1222 and an amplitude feature (e.g., PTT_{J}−A_{K}: r=0.72+/−0.05). These correlations may be readily interpreted in that PTT and amplitude features may represent DP and PP, respectively. SV may be regressed with T_{JJ} 1234 and A_{IJ}·PTT_{I}² 1236 (r=0.64+/−0.06). T_{JJ} 1234 may have been selected since it exhibits a positive correlation with SV in this work as stated above, while A_{IJ}·PTT_{I}² 1236 may be a meaningful surrogate of SV as stated earlier. CO may be regressed with T_{JJ} 1234 and PTT_{J} 1222 (r=0.76+/−0.04), which may be relevant in that T_{JJ} 1234 and PTT_{J} 1222 may represent heart rate and PP (which in general correlates with SV; also DP and PP varied in the same direction in response to the hemodynamic interventions considered in this work as shown in FIG. 7), respectively. TPR may be regressed with T_{JJ} 1234 and A_{JK}·PTT_{I}² 1238 (r=0.75+/−0.05) similarly to the whole-body BCG case.

### 4.4. Summarizing Remarks

In summary, the results obtained from this work provide several important implications. First, the characteristic features in the limb BCG have the potential for unobtrusive estimation of CV parameters. Indeed, for both the whole-body and armband BCG, the pair of as few as two features could achieve close correlation with CV parameters. Second, the characteristic features selected by the machine learning analysis (e.g., multivariate regression analysis) appeared to be largely interpretable (meaning that the selected characteristic features were to a large extent congruent with the physiological insights). Indeed, despite the fact that the machine learning analysis (e.g., multivariate regression analysis) conducted in this work was predominantly a data mining exercise, the majority of the characteristic features selected by the analysis are physiologically relevant and consistent with the findings derived from the mathematical model-based analysis of the BCG (see Sections 4.2 and 4.3 for details). Hence, the BCG features identified to exhibit close association with CV parameters in this work may be generalizable to other independent datasets. Third, PTT may make significant contributions in CV parameter estimation. Indeed, PTT was selected in all the bivariate regression analyses derived for BP (DP, PP, and SP) in this work. In comparison with our work in the Appendix in this application, that investigated the association between the characteristic features in the wrist BCG and BP (r=0.75 for both DP and SP on the average when three predictors were employed), this work achieves much higher correlation with less number of predictors (i.e., two) by including PTT. From this standpoint, it may be of interest to see the potential value of pulse arrival time (PAT) in further improving the association between the limb BCG and CV parameters. In fact, existing work suggests that PAT may serve as a good characteristic feature for SP as well as CV parameters via the pre-ejection period (which has implications on the heart contractility). One practical consideration may be that the use of PAT necessitates the measurement of the ECG, which generally requires conventional electrodes or two-handed user maneuvers. In this regard, accuracy-convenience trade-off may need to be made.

### Apparatus

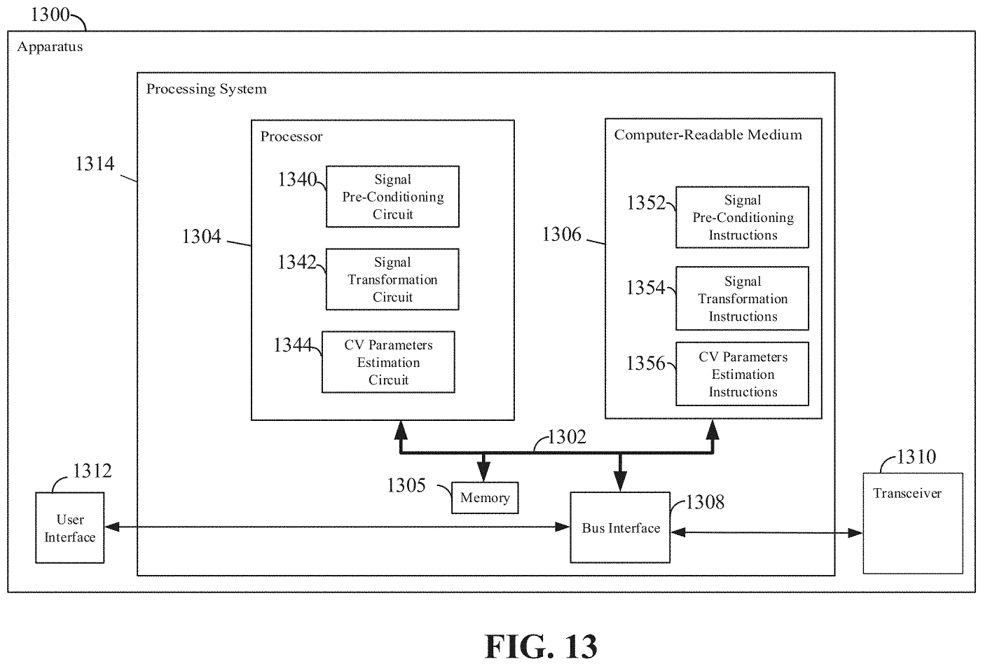

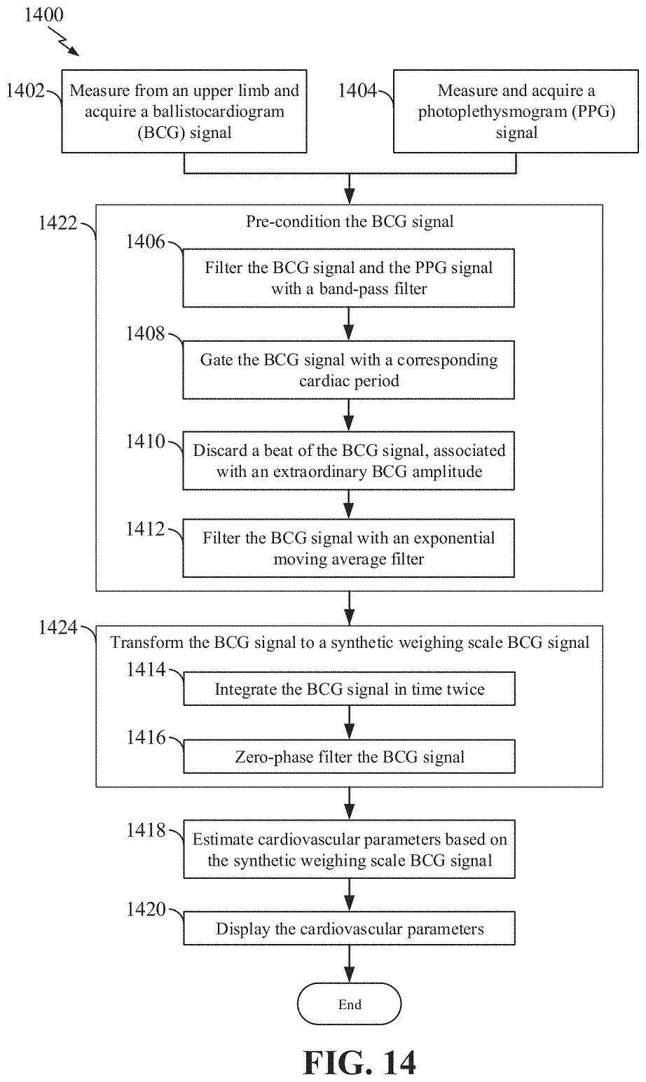

FIG. 13 is a block diagram illustrating an example of a hardware implementation for an apparatus 1300 employing a processing system 1314. The apparatus 1300 may include a processing system 1314 having one or more processors 1304. Examples of processors 1304 include microprocessors, microcontrollers, digital signal processors (DSPs), field programmable gate arrays (FPGAs), programmable logic devices (PLDs), state machines, gated logic, discrete hardware circuits, and other suitable hardware configured to perform the various functionality described throughout this disclosure. In various examples, the scheduling entity 700 may be configured to perform any one or more of the functions described herein. That is, the processor 1304, as utilized in an apparatus 700, may be configured (e.g., in coordination with the memory 1305) to implement any one or more of the processes and procedures described below and illustrated in FIG. 14.

The processing system 1314 may be implemented with a bus architecture, represented generally by the bus 1302. The bus 1302 may include any number of interconnecting buses and bridges depending on the specific application of the processing system 1314 and the overall design constraints. The bus 1302 communicatively couples together various circuits including one or more processors (represented generally by the processor 1304), a memory 1305, and computer-readable media (represented generally by the computer-readable medium 1306). The bus 1302 may also link various other circuits such as timing sources, peripherals, voltage regulators, and power management circuits, which are well known in the art, and therefore, will not be described any further. A bus interface 1308 provides an interface between the bus 1302 and a transceiver 1310. The transceiver 1310 provides a communication interface or means for communicating with various other apparatus over a transmission medium. Depending upon the nature of the apparatus, a user interface 1312 (e.g., keypad, display, speaker, microphone, joystick) may also be provided. Of course, such a user interface 1312 is optional, and some examples may omit it.

In some aspects of the disclosure, the processor 1304 may include a signal pre-conditioning circuitry 1340 configured (e.g., in coordination with the memory 1305) for various functions, including, e.g., filtering the BCG signal and the PPG signal with a band-pass filter, gating the BCG signal with a corresponding cardiac period, discarding a beat of the BCG signal, the beat associated with an amplitude of the BCG signal outside of a predetermined amplitude, and/or filtering the BCG signal with an exponential moving average filter. For example, the signal pre-conditioning circuitry 1340 may be configured to implement one or more of the functions described below in relation to FIG. 14, including, e.g., blocks 1406, 1408, 1410, 1412, and/or 1422. The processor 1304 may also include a signal transformation circuit 1342 configured (e.g., in coordination with the memory 1305) for various functions, including, e.g., integrating the BCG signal in time twice and/or zero-phase filtering the BCG signal. For example, the signal transformation circuit 1342 may be configured to implement one or more of the functions described below in relation to FIG. 14, including, e.g., blocks 1414, 1416, and/or 1424. The processor 1304 may also include a CV parameters estimation circuit 1344 configured (e.g., in coordination with the memory 1305) for various functions, including, e.g., estimating CV parameters based on the synthetic whole-body BCG signal, and/or estimating DP, PP, SP, SV, CP, and/or TRR. For example, the CV parameters estimation circuit 1344 may be configured to implement one or more of the functions described below in relation to FIG. 14, including, e.g., block 1418.

The processor 1304 is responsible for managing the bus 1302 and general processing, including the execution of software stored on the computer-readable medium 1306. The software, when executed by the processor 1304, causes the processing system 1314 to perform the various functions described below for any particular apparatus. The processor 1304 may also use the computer-readable medium 1306 and the memory 1305 for storing data that the processor 1304 manipulates when executing software.

One or more processors 1304 in the processing system may execute software. Software shall be construed broadly to mean instructions, instruction sets, code, code segments, program code, programs, subprograms, software modules, applications, software applications, software packages, routines, subroutines, objects, executables, threads of execution, procedures, functions, etc., whether referred to as software, firmware, middleware, microcode, hardware description language, or otherwise. The software may reside on a computer-readable medium 1306. The computer-readable medium 1306 may be a non-transitory computer-readable medium. A non-transitory computer-readable medium includes, by way of example, a magnetic storage device (e.g., hard disk, floppy disk, magnetic strip), an optical disk (e.g., a compact disc (CD) or a digital versatile disc (DVD)), a smart card, a flash memory device (e.g., a card, a stick, or a key drive), a random access memory (RAM), a read only memory (ROM), a programmable ROM (PROM), an erasable PROM (EPROM), an electrically erasable PROM (EEPROM), a register, a removable disk, and any other suitable medium for storing software and/or instructions that may be accessed and read by a computer. The computer-readable medium 1306 may reside in the processing system 1314, external to the processing system 1314, or distributed across multiple entities including the processing system 1314. The computer-readable medium 1306 may be embodied in a computer program product. By way of example, a computer program product may include a computer-readable medium in packaging materials. Those skilled in the art will recognize how best to implement the described functionality presented throughout this disclosure depending on the particular application and the overall design constraints imposed on the overall system.

In one or more examples, the computer-readable storage medium 1306 may store computer-executable code that includes signal pre-conditioning instructions 1352 that configure an apparatus 1300 for various functions. For example, the signal pre-conditioning instructions 1352 may be configured to cause an apparatus 1300 to implement one or more of the functions described below in relation to FIG. 14, including, e.g., blocks 1406, 1408, 1410, 1412, and/or 1422. The signal transformation instructions 1354 may further be configured to cause an apparatus 1300 to implement one or more of the functions described below in relation to FIG. 14, including, e.g., blocks 1414, 1416 and/or 1424. The CV parameters estimation instructions 1356 may further be configured to cause an apparatus 1300 to implement one or more of the functions described below in relation to FIG. 14, including, e.g., blocks 1418.

Of course, in the above examples, the circuitry included in the processor 1304 is merely provided as an example, and other means for carrying out the described functions may be included within various aspects of the present disclosure, including but not limited to the instructions stored in the computer-readable storage medium 1306, or any other suitable apparatus or means described in any one of the FIGS. 1-12, and utilizing, for example, the processes and/or algorithms described herein in relation to FIG. 14.

FIG. 14 is a flow chart illustrating an exemplary process 1400 for estimating CV parameters based on an armband BCG signal in accordance with some aspects of the present disclosure. As described below, a particular implementation may omit some or all illustrated features, and may not require some illustrated features to implement all embodiments. In some examples, the apparatus 1300 illustrated in FIG. 13 may be configured to carry out the process 1400. In some examples, any suitable apparatus or means for carrying out the functions or algorithm described below may carry out the process 1300.

At block 1402, an apparatus may acquire a BCG signal from a BCG sensor. The BCG sensor might be a high-resolution accelerometer embedded in an armband equipped with a wireless amplifier. The accelerometer is not limited to be embedded in an armband, nor to use in humans (veterinary uses may also be accomplished via process 1400). It could be a wrist BCG instrumented using an accelerometer embedded in a wristband equipped with a wireless amplifier, could be an electrode attached via a suitable adhesive, or another device having a housing or other configuration suitable to holding an accelerometer or similar motion-based sensor against a limb. The sensor may be attached anywhere in an upper limb to measure a BCG signal. Alternatively, with suitable adjustments to the BCG characteristic measurements and CV correlations (described below), the sensor may be attached to lower limbs as well. The BCG signal may be measured as the body movement in response to the blood ejected by the heart (as such, various types of accelerometers are contemplated, as well as related sensors that measure movement (such as, for example, electrodes or electrical contacts generating a signal based upon their separation). The BCG signal may be measured through several hemodynamic interventions. For example, the hemodynamic interventions may be four states: a cold pressor intervention in which the subject immerses a free hand in ice water for a suitable period of time; a mental arithmetic intervention in which the subject repeatedly adds numbers for a suitable period of time; a slow breathing intervention in which the subject takes deep and slow breaths for a suitable period of time; and a breath holding intervention in which the subject holds breath after normal exhalation. Between two interventions, there may be a rest state for a suitable period of time. However, it should be appreciated that the interventions described above are mere examples to construct the machine learning regression models. Once such machine learning regression models are available, CV parameters can be readily estimated by measuring the BCG signal, extracting the features therein, and inputting them to the machine learning regression models.

At block 1404, an apparatus may acquire a PPG signal from a finger clip sensor. However, the sensor is not necessarily a finger clip sensor. It should be appreciated that the sensor may be any suitable type of sensor which measure a PPG signal or related signal. The PPG signal may be obtained by using a pulse oximeter which illuminates the skin and measures changes in light absorption. Thus, the PPG signal may indicate blood volume changes in the microvascular bed of tissue. In another embodiment, a mobile device may be configured to acquire a PPG signal, with a lead, Bluetooth, or other connection to an accessory comprising an accelerometer for a BCG signal. In one embodiment, the PPG signal and BCG signal may be acquired from the same sensor device, e.g., having both motion and optical sensors.

At block 1422, the BCG signal may optionally be modulated in a signal pre-conditioning procedure for preparing transformation of the BCG signal to a synthetic whole-body BCG signal. At block 1406, the signal pre-conditioning procedure 1422 may begin with zero-phase filtering the BCG signal and the PPG signal. The zero-phase filtering may use a band-pass filter. For example, the BCG and PPG may be smoothed by a 2^{nd}-order Butterworth band-pass filter with a pass band of 0.5˜10 Hz. However, the type of filter and the pass band should not be limited to the 2^{nd}-order Butterworth band-pass filter with the pass band of 0.5˜10 Hz. It could by any suitable type of filter and a suitable pass band to smooth the BCG and PPG signals. Step 1422 may be desirable in instances wherein compatibility or comparability with a whole-body BCG measurement is desirable. However, the steps described below for determining CV characteristics from a whole-body BCG signal can be adapted for determining CV characteristics directly from a limb-based BCG signal. Alternatively, rather than utilizing a synthetic whole-body BCG signal, the steps below could also be performed on true whole-body BCG signals obtained from, for example, a weight/scale based sensor.

At block 1408, the filtered BCG signal and PPG signal may be gated with a time instant. The time instant may correspond to 10% of cardiac period before any fiducial point indicating the beginning of the cardiac period (e.g., the R wave in the ECG signal). However, the time instant may not be limited to correspond to 10% of cardiac period. It could be any suitable time.

At block 1410, the low-quality BCG signal may be discarded. One example to discard the low-quality BCG signal is by calculating the amplitudes associated with all the BCG beats of the BCG signal, and (ii) discarding the beats associated with extraordinarily large or small BCG amplitude. For example, the extraordinarily large or small BCG amplitude may be outside of 3 scaled median absolute deviations (with the scaling factor of 1.4826) around the median amplitude. The extraordinarily large or small BCG amplitude may also be determined by any other suitable factor.

At block 1412, the BCG signal may be smoothed using a filter to suppress the adverse impact of motion artifacts. The filter may be a 10-beat exponential moving average filter. However, it should be appreciated that the type of filter is not limited to the 10-beat exponential moving average filter. It could be any suitable filter to suppress the adverse impact of motion artifacts.

At block 1424, the BCG signal may be transformed to a synthetic whole-body BCG signal. The BCG signal from an upper limb without this transformation is hard to find the relationship with CV parameter. However, the transformed BCG signal (which is the synthetic whole-body BCG signal) is an equivalent whole-body BCG. Thus, the synthetic whole-body BCG signal may exhibit close correlation with the CV parameters as a whole-body BCG does. To transform the BCG signal to the synthetic whole-body BCG signal, the BCG signal may be integrated in time twice at block 1414 and zero-phase filtered at block 1416.

In particular, at block 1414, the BCG signal may be integrated in time twice using the trapezoidal method to yield the synthetic whole-body BCG signal. Given the armband BCG signal samples BCG_{A} (k), k=1, . . . , N, it is integrated once to yield an intermediate signal BCG_{V}(k), k=1, . . . , N, where BCG_{V}(k) is given by

$$B C {G}_{V} ( k ) = \frac{Δ t}{2} [ B C {G}_{A} ( 1 ) + 2 B C {G}_{A} ( 2 ) + … + 2 {BCG}_{A} ( k - 1 ) + B C {G}_{A} ( k ) ] .$$

Then, BCG_{V}(k), k=1, . . . , N is integrated once again to yield BCG_{W}(k), k=1, . . . , N, where BCG_{W}(k) is given by

$$B C {G}_{W} ( k ) = \frac{Δ t}{2} [ B C {G}_{V} ( 1 ) + 2 B C {G}_{V} ( 2 ) + … + 2 {BCG}_{V} ( k - 1 ) + B C {G}_{V} ( k ) ] .$$

However, it should be appreciated that the integration method is not limited to the trapezoidal method. It could be any other suitable method to integrate the BCG signal in time twice and produce the synthetic whole-body BCG signal.

Then, at block 1416, the synthetic whole-body BCG signal may be zero-phase filtered. The zero-phase filtering may use a 4^{th}-order Butterworth high-pass filter to remove the low-frequency drift therein. However, it should be appreciated that the filter could be any other suitable filter to remove the low-frequency drift in the synthetic whole-body BCG signal. The cut-off frequency of the filter may be determined empirically as the average of the 2^{nd} and 3^{rd} peaks in the BCG power spectrum. Alternatively, the cut-off frequency may be computed from the heart rate as 2.5 times the heart rate, since the spectral peaks in the BCG represent the heart rate and its harmonics.

At block 1418, based on the synthetic whole-body BCG signal, CV parameters may be estimated. The synthetic whole-body BCG signal may include a periodic waveform having a first trough, a first peak, and a second trough. The PPG signal may have a PPG wave form having a PPG foot. The PPG foot may be determined using the intersecting tangent method. The first trough may be predominantly associated with an ascending aortic blood pressure (BP), the first peak may be predominantly associated with a descending aortic BP, and the second trough may be predominantly associated with the ascending aortic BP and the descending aortic BP.

The CV parameters may include a diastolic BP (DP), a pulse BP (PP), a systolic BP (SP), a stroke volume (SV), a cardiac output (CO), or a total peripheral resistance (TRR). These CV parameters may be related to the characteristics features in the BCG signal via machine learning regression models (e.g., linear regression models). For example, if multivariate linear regression technique is used, DP may be estimated based on a PTT(I) and an A(J): DP=k_{1,DP}PTT(I)+k_{2,DP}A(J)+k_{3,DP}, the PP is estimated based on the PTT(I) and a T(JJ): PP=k_{1,PP}PTT(I)+k_{2,PP}T(B)+k_{3,PP}, the SP is estimated based on an A(JK) and an A(IJ)·PTT(I)²: SP=k_{1,SP}A(JK)+k_{2,SP}A(IJ)·PTT(I)²+k_{3,SP}, the SV is estimated based on the T(JJ) and the A(IJ)·PTT(I)²: SV=k_{1,SV}T(JJ)+k_{2,SV}A(IJ)·PTT(I)²+k_{3,SV}, the CO is estimated based on the T(JJ) and a PTT(J): CO=k_{1,CO}T(JJ)+k_{2,CO}PTT(J)+k_{3,CO}, and/or the TRR is estimated based on the T(JJ) and an A(JK)·PTT(I)²: TPR=k_{1,TPR}T(JJ)+k_{2,TPR}A(JK)·PTT(I)²+k_{3,TPR}. Here, the PTT(I) is a time interval between the first trough and the PPG foot, the A(J) is an amplitude at the first trough, the T(JJ) is a time interval between the first peak of the BCG signal and another peak of a second BCG signal, the A(JK) is an amplitude difference between the first peak and the second trough, the A(IJ) is difference between the first peak and the second trough times, wherein PTT(I)² is a square time interval between the first trough and the PTT trough, and the PTT(J) is a time interval between the first peak and the PPG foot. The coefficients k_{1,X}, k_{2,X}, and k_{3,X} in the linear regression models above (where X=DP, PP, SP, SV, CO, and TPR) may be derived using the reference CV parameters and the BCG signals collected from each subject or from many subjects using, e.g., standard linear least squares minimization technique. The same regression technique may also be used to correlate CV parameters to synthetic whole-body BCG signals, true whole-body BCG signals, or limb-based BCG signals. In addition, other machine learning analysis and regression techniques (e.g., partial least squares, support vector regression, and neural networks) may likewise be used to relate CV parameters to synthetic whole-body BCG signals, true whole-body BCG signals, or limb-based BCG signals.

At block 1420, the CV parameters may be displayed. It may be values of CV parameters, a history of CV parameters for a subject for a predetermined period of time, which indicates the changes of the CV parameters in the predetermined period of time. In addition, using a deep learning algorithm, the CV parameters and changes of the CV parameters may be inputted to produce a possibility of CV diseases and suggested treatments.

This disclosure presents several aspects for estimating CV parameters based on an armband or whole-body BCG signal with reference to an exemplary implementation. As those skilled in the art will readily appreciate, various aspects described throughout this disclosure may be extended to other systems, apparatuses, and modules.

The present disclosure uses the word “exemplary” to mean “serving as an example, instance, or illustration.” Any implementation or aspect described herein as “exemplary” is not necessarily to be construed as preferred or advantageous over other aspects of the disclosure. Likewise, the term “aspects” does not require that all aspects of the disclosure include the discussed feature, advantage or mode of operation. The present disclosure uses the term “coupled” to refer to a direct or indirect coupling between two objects. For example, if object A physically touches object B, and object B touches object C, then objects A and C may still be considered coupled to one another—even if they do not directly physically touch each other. For instance, a first object may be coupled to a second object even though the first object is never directly physically in contact with the second object. The present disclosure uses the terms “circuit” and “circuitry” broadly, to include both hardware implementations of electrical devices and conductors that, when connected and configured, enable the performance of the functions described in the present disclosure, without limitation as to the type of electronic circuits, as well as software implementations of information and instructions that, when executed by a processor, enable the performance of the functions described in the present disclosure.

One or more of the components, steps, features and/or functions illustrated in FIGS. 1-14 may be rearranged and/or combined into a single component, step, feature or function or embodied in several components, steps, or functions. Additional elements, components, steps, and/or functions may also be added without departing from novel features disclosed herein. The apparatus, devices, and/or components illustrated in FIGS. 1-14 may be configured to perform one or more of the methods, features, or steps described herein. The novel algorithms described herein may also be efficiently implemented in software and/or embedded in hardware.

It is to be understood that the specific order or hierarchy of steps in the methods disclosed is an illustration of exemplary processes. Based upon design preferences, it is understood that the specific order or hierarchy of steps in the methods may be rearranged.

What is claimed is:

1. An apparatus for estimating cardiovascular parameters, comprising:
a ballistocardiogram (BCG) sensor for producing a BCG signal of a patient from a limb of the patient;
a processor;
a first memory communicatively coupled to the processor and having a set of software instructions stored thereon which, when executed by the processor, cause the processor to:
receive BCG data reflecting the signal produced by the BCG sensor;
estimate cardiovascular parameters of the patient based on the BCG data; and
send the cardiovascular parameters to at least one of a second memory or a display.

2. The apparatus of claim 1, wherein the BCG sensor comprises a high-resolution accelerometer attached on an upper limb of the user.

3. The apparatus of claim 1, wherein the BCG data comprises synthetic whole-body BCG data generated from the BCG signal produced by the sensor.

4. The apparatus of claim 1, further comprising:
a photoplethysmogram (PPG) sensor for producing a PPG signal.

5. The apparatus of claim 4, wherein software instructions further cause the processor to:
pre-condition the BCG signal,
wherein the pre-conditioning the BCG signal comprises:
filtering the BCG signal and the PPG signal with a band-pass filter;
gating the BCG signal with a corresponding cardiac period;
discarding a beat of the BCG signal, the beat associated with an amplitude of the BCG signal outside of a predetermined amplitude; and
filtering the BCG signal with an exponential moving average filter.

6. The apparatus of claim 4, wherein the BCG signal comprises a periodic waveform having a first trough, a first peak, and a second trough, and
wherein the PPG signal has a periodic PPG waveform having a PPG foot.

7. The apparatus of claim 6, wherein the first trough is predominantly associated with an ascending aortic blood pressure (BP), the first peak is predominantly associated with a descending aortic BP, and the second trough is predominantly associated with the ascending aortic BP and the descending aortic BP.

8. The apparatus of claim 6, wherein the cardiovascular parameters comprise at least one of: a diastolic BP (DP), a pulse BP (PP), a systolic BP (SP), a stroke volume (SV), a cardiac output (CO), or a total peripheral resistance (TRR).

9. The apparatus of claim 8,
wherein the DP is estimated based on a time interval between the first trough and the PPG foot, and an amplitude at the first trough,
wherein the PP is estimated based on the time interval between the first trough and the PPG foot, and a time interval between the first peak of the BCG signal and another peak of a second BCG signal,
wherein the SP is estimated based on an amplitude difference between the first peak and the second trough, a difference between the first peak and the second trough times, and a square time interval between the first trough and the PTT trough,
wherein the SV is estimated based on the time interval between the first peak of the BCG signal and the another peak of the second BCG signal, the difference between the first peak and the second trough times, and the square time interval between the first trough and the PTT trough,
wherein the CO is estimated based on the time interval between the first peak of the BCG signal and the another peak of the second BCG signal, and a time interval between the first peak and the PPG foot,
wherein the TRR is estimated based on the time interval between the first peak of the BCG signal and the another peak of the second BCG signal, the amplitude difference between the first peak and the second trough, and the square time interval between the first trough and the PTT trough.

10. A system for estimating cardiovascular parameters, comprising:
a ballistocardiogram (BCG) sensor for producing a BCG signal of a patient from a limb of the patient;
a processor;
a first memory communicatively coupled to the processor and having a set of software instructions stored thereon which, when executed by the processor, cause the processor to:
receive BCG data reflecting the signal produced by the BCG sensor;
estimate cardiovascular parameters of the patient based on the BCG data; and
send the cardiovascular parameters to at least one of a second memory or a display.

11. The system of claim 10, wherein the BCG sensor comprises a high-resolution accelerometer attached on an upper limb of the user.

12. The system of claim 10, wherein the BCG data comprises synthetic whole-body BCG data generated from the BCG signal produced by the sensor.

13. The system of claim 10, further comprising:
a photoplethysmogram (PPG) sensor for producing a PPG signal.

14. The system of claim 13, wherein software instructions further cause the processor to:
pre-condition the BCG signal,
wherein the pre-conditioning the BCG signal comprises:
filtering the BCG signal and the PPG signal with a band-pass filter;
gating the BCG signal with a corresponding cardiac period;
discarding a beat of the BCG signal, the beat associated with an amplitude of the BCG signal outside of a predetermined amplitude; and
filtering the BCG signal with an exponential moving average filter.

15. The system of claim 13, wherein the BCG signal comprises a periodic waveform having a first trough, a first peak, and a second trough, and
wherein the PPG signal has a periodic PPG waveform having a PPG foot.

16. The system of claim 15, wherein the first trough is predominantly associated with an ascending aortic blood pressure (BP), the first peak is predominantly associated with a descending aortic BP, and the second trough is predominantly associated with the ascending aortic BP and the descending aortic BP.

17. The system of claim 15, wherein the cardiovascular parameters comprise at least one of: a diastolic BP (DP), a pulse BP (PP), a systolic BP (SP), a stroke volume (SV), a cardiac output (CO), or a total peripheral resistance (TRR).

18. The system of claim 17,
wherein the DP is estimated based on a time interval between the first trough and the PPG foot, and an amplitude at the first trough,
wherein the PP is estimated based on the time interval between the first trough and the PPG foot, and a time interval between the first peak of the BCG signal and another peak of a second BCG signal,
wherein the SP is estimated based on an amplitude difference between the first peak and the second trough, a difference between the first peak and the second trough times, and a square time interval between the first trough and the PTT trough,
wherein the SV is estimated based on the time interval between the first peak of the BCG signal and the another peak of the second BCG signal, the difference between the first peak and the second trough times, and the square time interval between the first trough and the PTT trough,
wherein the CO is estimated based on the time interval between the first peak of the BCG signal and the another peak of the second BCG signal, and a time interval between the first peak and the PPG foot,
wherein the TRR is estimated based on the time interval between the first peak of the BCG signal and the another peak of the second BCG signal, the amplitude difference between the first peak and the second trough, and the square time interval between the first trough and the PTT trough.

19. A method for estimating cardiovascular parameters, comprising:
receive ballistocardiogram (BCG) data reflecting a BCG signal of a patient from a limb of the patient, the BCG signal produced by a BCG sensor;
estimate cardiovascular parameters of the patient based on the BCG data; and
send the cardiovascular parameters to at least one of a memory or a display.

20. The method of claim 19, wherein the BCG sensor comprises a high-resolution accelerometer attached on an upper limb of the user.

21. The method of claim 19, wherein the BCG data comprises synthetic whole-body BCG data generated from the BCG signal produced by the sensor.

22. The method of claim 19, further comprising:
receiving a PPG signal from a photoplethysmogram (PPG) sensor.

23. The method of claim 22, further comprising:
pre-condition the BCG signal,
wherein the pre-conditioning the BCG signal comprises:
filtering the BCG signal and the PPG signal with a band-pass filter;
gating the BCG signal with a corresponding cardiac period;
discarding a beat of the BCG signal, the beat associated with an amplitude of the BCG signal outside of a predetermined amplitude; and
filtering the BCG signal with an exponential moving average filter.

24. The method of claim 22, wherein the BCG signal comprises a periodic waveform having a first trough, a first peak, and a second trough, and
wherein the PPG signal has a periodic PPG waveform having a PPG foot.

25. The method of claim 24, wherein the first trough is predominantly associated with an ascending aortic blood pressure (BP), the first peak is predominantly associated with a descending aortic BP, and the second trough is predominantly associated with the ascending aortic BP and the descending aortic BP.

26. The method of claim 24, wherein the cardiovascular parameters comprise at least one of: a diastolic BP (DP), a pulse BP (PP), a systolic BP (SP), a stroke volume (SV), a cardiac output (CO), or a total peripheral resistance (TRR).

27. The method of claim 26,
wherein the DP is estimated based on a time interval between the first trough and the PPG foot, and an amplitude at the first trough,
wherein the PP is estimated based on the time interval between the first trough and the PPG foot, and a time interval between the first peak of the BCG signal and another peak of a second BCG signal,
wherein the SP is estimated based on an amplitude difference between the first peak and the second trough, a difference between the first peak and the second trough times, and a square time interval between the first trough and the PTT trough,
wherein the SV is estimated based on the time interval between the first peak of the BCG signal and the another peak of the second BCG signal, the difference between the first peak and the second trough times, and the square time interval between the first trough and the PTT trough,
wherein the CO is estimated based on the time interval between the first peak of the BCG signal and the another peak of the second BCG signal, and a time interval between the first peak and the PPG foot,
wherein the TRR is estimated based on the time interval between the first peak of the BCG signal and the another peak of the second BCG signal, the amplitude difference between the first peak and the second trough, and the square time interval between the first trough and the PTT trough.
